(19) United States
(12) Patent Application Publication
(10) Pub. No.: US 2025/0339710 A1
(43) Pub. Date: Nov. 6, 2025

(54) METHODS, APPARATUSES, DEVICES, AND STORAGE MEDIA FOR GENERATING ARC RADIOTHERAPY PLANS

(71) Applicant: MEVION MEDICAL EQUIPMENT CO., LTD., Suzhou (CN)

(72) Inventors: Xuemin BAI, Suzhou (CN); Yuan YOU, Suzhou (CN)

(73) Assignee: MEVION MEDICAL EQUIPMENT CO., LTD., Suzhou (CN)

(21) Appl. No.: 19/266,139

(22) Filed: Jul. 10, 2025

Related U.S. Application Data
(63) Continuation-in-part of application No. PCT/CN2024/070587, filed on Jan. 4, 2024.

(30) Foreign Application Priority Data
Jan. 16, 2023 (CN) 202310066061.7

(51) Int. Cl. A61N 5/10; G16H 20/40

(52) U.S. Cl. CPC A61N 5/103; G16H 20/40

(57) ABSTRACT

A method, apparatus, device, and storage medium for generating an arc radiotherapy plan are provided. The method includes obtaining a reference beam set of each scanning point in a target volume; determining a structure contribution and a target volume contribution of each reference beam and determining an importance factor based on the two; constructing a particle source selection function by combining the importance factor, the reference beam set, and a complexity control parameter and performing optimization solution to obtain a target beam set; generating an arc radiotherapy plan based on the target beam set, the arc radiotherapy plan specifying in detail beam energy and monitor units of each control point on an arc scanning path. With the arc radiotherapy plan, a particle accelerator delivers the target beam precisely to the target volume. The method comprehensively considers beam spot data and the importance factor, improves accuracy and adaptability of radiotherapy plan.

### CROSS-REFERENCE TO RELATED APPLICATIONS

This application is a Continuation-in-part of International Application No. PCT/CN2024/070587, filed on Jan. 4, 2024, which claims priority to Chinese Patent Application No. 202310066061.7, filed on Jan. 16, 2023, the entire contents of each of which are hereby incorporated by reference.

### TECHNICAL FIELD

The present disclosure relates to the field of radiation therapy technology, and in particular, to methods, apparatuses, devices, and storage media for generating arc radiotherapy plans.

### BACKGROUND

Particle arc therapy (PAT) is a radiation treatment method in which beams are continuously delivered or at discrete control points as a treatment gantry rotates around the patient. Unlike intensity-modulated particle therapy with multiple radiation fields, PAT introduces irradiation paths with increased angular dimensionality under controlled precision. Compared to fixed-angle intensity-modulated particle radiotherapy, this approach offers superior angular coverage and potential for improved dose homogeneity, which markedly enhances the efficiency of treatment plan delivery and improves the quality of the treatment plan.

Currently, numerous researchers have investigated PAT and proposed various particle arc treatment plans. Most studies focus on reducing the switching time of the energy layer of particle accelerators by minimizing the count of energy layers used, thereby ensuring the clinical delivery efficiency of treatment plans.

However, some particle therapy systems use a range shifter to adjust the energy (or range) of the particle beam, where the switching time of the energy layer (range) may be reduced from seconds to milliseconds. For such systems, PAT does not significantly affect the efficiency of clinical plan delivery. Therefore, research should be focused on enhancing the quality of the treatment plan.

### SUMMARY

One or more embodiments of the present disclosure provide a method, apparatus, device, and storage medium for generating an arc radiotherapy plan to solve the problem of the poor quality of the associated radiotherapy plan to improve the quality of the radiotherapy plan.

One or more embodiments of the present disclosure provide a method for generating an arc radiotherapy plan. The method may include obtaining a plurality of reference beam sets corresponding to a plurality of scanning points in a target volume; for a reference beam in one of the plurality of reference beam sets, obtaining a structure contribution of the reference beam corresponding to a critical structure and a target volume contribution of the reference beam corresponding to the target volume, and determining an importance factor of the reference beam based on the structure contribution and the target volume contribution; obtaining a plurality of target beam sets corresponding to the plurality of scanning points based on a plurality of importance factors corresponding to a plurality of reference beams, the plurality of reference beam sets, and a complexity control parameter; generating the arc radiotherapy plan based on a plurality of target beams in the plurality of target beam sets, where the arc radiotherapy plan includes beam energy and monitor units for a plurality of control points within an arc angle range; and controlling a particle accelerator to deliver the plurality of target beams to the target volume based on the beam energy and the monitor units.

One or more embodiments of the present disclosure provide an apparatus for generating an arc radiotherapy plan. The apparatus may include a storage unit and a processing unit. The storage unit may be configured to store a computer program. The processing unit may be configured to invoke the computer program and execute and execute the computer program to implement the method for generating the arc radiotherapy plan.

One or more embodiments of the present disclosure provide an electronic device. The electronic device may include at least one processor, and a memory communicatively coupled to the at least one processor. The memory may store a computer program executable by the at least one processor, and the computer program may, when executed by the at least one processor, cause the at least one processor to perform the method for generating the arc radiotherapy plan as described in any embodiment of the present disclosure, or to implement functions of the apparatus for generating the arc radiotherapy plan as described in any embodiment of the present disclosure.

One or more embodiments of the present disclosure provide a non-transitory computer-readable storage medium storing computer instructions. The computer instructions may be used to cause a processor to perform the method for generating the arc radiotherapy plan as described in any one of the present disclosures, or to implement functions of the apparatus for generating the arc radiotherapy plan as described in any embodiment of the present disclosure.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is a diagram illustrating an application scenario of a system for generating an arc radiotherapy plan according to some embodiments of the present disclosure;

FIG. 2 is a flowchart illustrating a process for generating an arc radiotherapy plan according to some embodiments of the present disclosure;

FIG. 3 is a flowchart illustrating a process for generating an arc radiotherapy plan according to some other embodiments of the present disclosure;

FIG. 4 is a schematic diagram illustrating a comparison between beam layouts according to some embodiments of the present disclosure;

FIG. 5 is a flowchart illustrating a process for optimizing an arc radiotherapy plan according to some embodiments of the present disclosure;

FIG. 6 is a flowchart illustrating an embodiment of a process for generating an arc radiotherapy plan according to some embodiments of the present disclosure;

FIG. 7 is a schematic diagram illustrating an apparatus for generating an arc radiotherapy plan according to some embodiments of the present disclosure; and

FIG. 8 is a schematic diagram illustrating an electronic device according to some embodiments of the present disclosure.

### DETAILED DESCRIPTION

The accompanying drawings, which are required to be used in the description of the embodiments, are briefly described below. The accompanying drawings do not represent the entirety of the embodiments.

As used herein, “system”, “device”, “unit” and/or “module” is a manner used to distinguish different components, elements, parts, sections, or assemblies at different levels. However, if other words serve the same purpose, the words may be replaced by other expressions.

As shown in the present disclosure and claims, the words “one”, “a”, “a kind” and/or “the” are not especially singular but may include the plural unless the context expressly suggests otherwise. In general, the terms “comprise”, “comprises”, “comprising”, “include”, “includes”, and/or “including”, merely prompt to include operations and elements that have been clearly identified, and these operations and elements do not constitute an exclusive listing. The methods or devices may also include other operations or elements.

When describing the operations performed in the embodiments of the present disclosure in step-by-step instructions, the order of the steps is interchangeable if not otherwise specified, the steps are omissible, and other steps may be included in the operation.

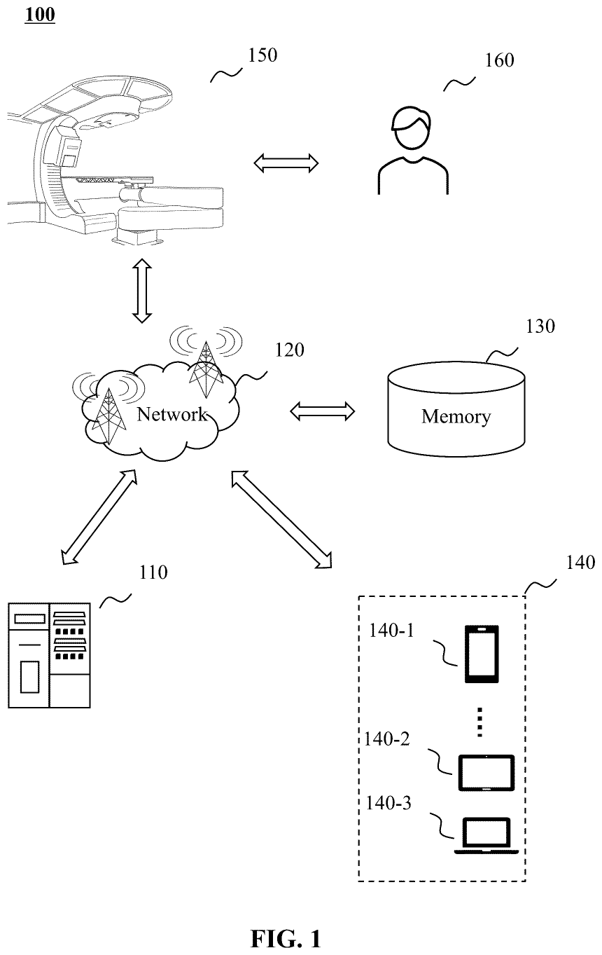

FIG. 1 is a diagram illustrating an application scenario of a system for generating an arc radiotherapy plan according to some embodiments of the present disclosure. An application scenario 100 of a system for generating an arc radiotherapy plan may include a processor 110, a network 120, a memory 130, a terminal device 140, a treatment machine 150, and a target object 160.

The processor 110 may be a variety of general-purpose and/or specialized processing components with processing and computational capabilities. The processor 110 includes but is not limited to, a central processing unit (CPU), a graphics processing unit (GPU), various specialized artificial intelligence (AI) computing chips, various processors running machine learning model algorithms, a digital signal processor (DSP), and any suitable processors, controllers, microcontrollers, or the like. The processor 110 performs various processes and operations described below, such as a process for generating an arc radiotherapy plan.

The network 120 refers to a communication network that may be configured to connect components in a system for transmission and sharing of data. The network 120 may be wired (e.g., Ethernet) or wireless (e.g., Wi-Fi, 4G, 5G, etc.).

The memory 130 refers to a component for storing data, which may be a hard disk, a solid-state drive, or other types of storage medium. The memory 130 is configured to hold data radiotherapy plans, beam parameters, or the like.

The terminal device 140 refers to a device used by a user, which may be configured as a smartphone 140-1, a tablet 140-2, a laptop 140-3, etc. The user may be a physician.

The treatment machine 150 refers to a medical device used to perform radiotherapy.

During an arc radiotherapy procedure, the treatment machine 150 may include a particle accelerator, a rotating gantry, a snout, and a treatment bed.

The particle accelerator accelerates charged particles (e.g., electrons) by an electromagnetic field and is used for particle therapy, such as proton therapy or heavy ion therapy.

During particle arc therapy, the therapy system typically consists of a particle accelerator (such as a synchrotron or cyclotron), a beam transport line, a rotating gantry, a snout, and a treatment bed. The particle accelerator generates high-energy proton beams, which are usually delivered via the beam transport line to the snout mounted on the rotating gantry. Alternatively, the particle accelerator may be directly mounted on the rotating gantry, rotating along with the rotating gantry to provide beams to the snout. The rotating gantry may revolve around (commonly 180° or 360°) the central axis of the target object to enable beam delivery from a plurality of angles. Each angle corresponds to a control point, where the delivered beam may have varying energy and dose to create an arc-shaped dose distribution covering the target volume. The target object remains stationary on the treatment bed, which may be adjusted as needed to facilitate precise beam delivery.

The target object 160 refers to an individual undergoing radiotherapy with one or more target volumes present in the body that require radiation treatment by the treatment machine 150. The target volume refers to a region of the target object that requires radiation treatment. For example, if the target object is a patient with a liver tumor, the target volume is a liver tumor region, etc.

In some embodiments, the processor 110 may be communicatively connected via the network 120 to other components in the application scenario 100 of the system for generating the arc radiotherapy plan (e.g., the memory 130, the terminal device 140, and the treatment machine 150), for data storage, reading, command execution, and control of the treatment machine, etc.

In some embodiments, other components may be included in the application scenario of the system for generating the arc radiotherapy plan, such as data transmission interfaces, signaling methodologies and actuators, directional control components, sensors (e.g., positioning sensors, angular monitoring sensors, dosage monitoring devices, or energy sensors), or the like.

In some embodiments, the components may be separately configured or integrated. For example, the processor 110 may be integrated with the memory 130 in the terminal device 140, and the directional control components, signaling methodologies and actuators, sensors, or the like may be deployed in the treatment machine 150.

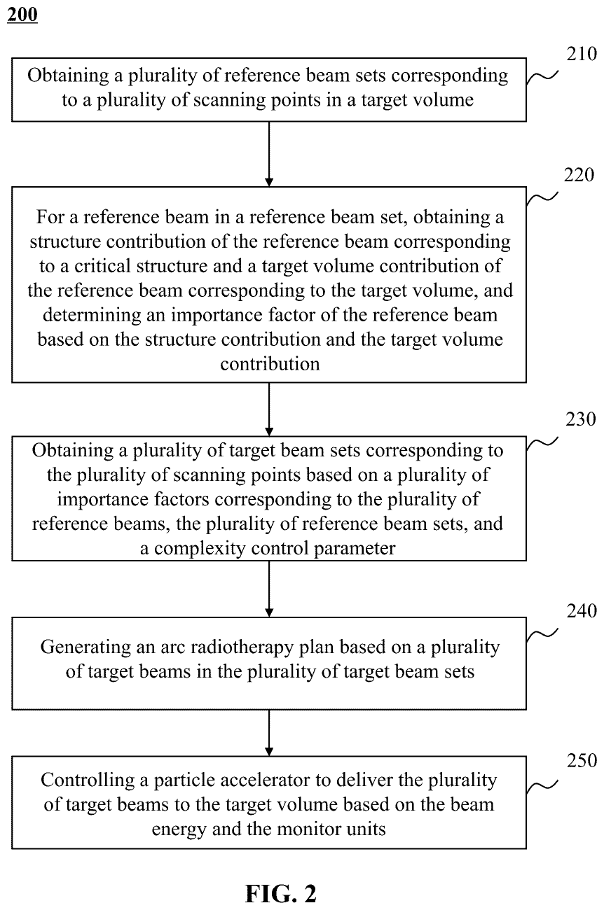

FIG. 2 is a flowchart illustrating a process for generating an arc radiotherapy plan according to some embodiments of the present disclosure. As shown in FIG. 2, process 200 includes the following operations. In some embodiments, the process 200 may be executed by a processor.

In 210, a plurality of reference beam sets corresponding to a plurality of scanning points are obtained in a target volume.

More descriptions regarding the target volume may be found in the related description in FIG. 1.

In some embodiments, the target volume may be pre-sketched and determined from image data by a user. The image data may be an X-ray computed tomography (CT) image or a magnetic resonance imaging (MRI) image, and the image type of the image data is not limited herein. The sketching operation may be manually performed by the user or automatically performed using a sketching tool. In some embodiments, there may be one or more target volumes.

The scanning points refer to discrete spatial points distributed along a surface or interior of the target volume. In some embodiments, there are a plurality of scanning points in a target volume, e.g., a scanning point may be denoted by k, k∈V, and V denotes the target volume.

In some embodiments, the processor may obtain simulated positioning CT data of the target object and three-dimensional contour data of the target volume and select each scanning point based on the simulated positioning CT data of the target object, the three-dimensional contour data, and scanning point distribution data summarized in the clinical radiotherapy plan. The three-dimensional contour data of the target volume may include voxel coordinates, a density value, and spatial position information of each voxel within the target volume, and the three-dimensional contour data may be obtained by the processor based on the image data through an image processing algorithm. The scanning point distribution data may be a scanning point distribution law. For example, a distance from any voxel of the target volume to the nearest scanning point is less than a preset distance (e.g., 3 mm), the plurality of scanning points are evenly distributed, and the plurality of scanning points may completely cover the entire target volume.

In some embodiments, the processor and/or a human may set the angle of the treatment bed, an angular range of the snout, and an angular spacing of the control points based on the actual needs of the clinical case and the physical limitations of the treatment machine. The control points are used to characterize points at which the beam source emits the beams, i.e., the control points are located within the angular range of the snout. Exemplarily, the angular range of the snout is 180° and the angular spacing of the control points is 2°, then a count of control points is 90.

A reference beam refers to a radiating beam used for a candidate target beam. In some embodiments, the beam source may emit a plurality of reference beams at each control point, a single reference beam may cover a plurality of scanning points, each scanning point may be covered by a plurality of reference beams, and different reference beams may contain different energies, different irradiation angles, different doses, etc. The reference beam is a virtual beam that is generated by calculation and is used as a candidate for determining a target beam that is ultimately used for radiotherapy.

In some embodiments, the processor may also set the beam spacing based on the actual needs and beam spot data of the reference beam. Too dense a beam spacing increases the calculation time, and too sparse a beam spacing affects the quality of the radiotherapy plan. Descriptions regarding the beam spot data may be found in the related description hereinafter.

A reference beam set refers to a set that includes a plurality of reference beams. In some embodiments, one scanning point corresponds to one reference beam set. For example, a reference beam set Jₖ denotes a set of all reference beams covering a k-th scanning point, with a reference beam j∈Jₖ. The reference beam set relates to the configuration of the beams in the arc radiotherapy plan, i.e., the selection of which beams are to be used for treating the corresponding scanning point.

In some embodiments, the target volume is irradiated by the snout from different angles, the snout may emit a plurality of beams with different energies and adjust target position of the beam by a scanning magnet within the radiation field, and the processor may employ a ray tracing algorithm to determine, based on the image data of the target object, the voxel coordinates through which each reference beam emitted by the snout respectively passes, and classify each reference beam based on the voxel coordinates corresponding to each reference beam, and determine a set of reference beams passing through a certain scanning point as a reference beam set corresponding to the scanning point, so as to obtain the reference beam sets corresponding to the scanning points in the target volume. The ray tracing algorithm requires computational parameters including the image data of the target object, a CT number (HU) to material density conversion table or a CT number (HU) to material stopping power reference table, an electron density control table, a beam angle, a particle energy spectrum parameter table, etc.

In 220, for a reference beam in a reference beam set, a structure contribution of the reference beam corresponding to a critical structure and a target volume contribution of the reference beam corresponding to the target volume are obtained, and an importance factor of the reference beam is determined based on the structure contribution and the target volume contribution.

The critical structure refers to an organ or tissue that is susceptible to injury by radiation beams during the performance of radiotherapy. In some embodiments, the critical structure may be a healthy organ or sensitive tissue close to the target volume. There may be one or more critical structures.

The structure contribution refers to a metric used to measure a risk of damage to a single critical structure from a single reference beam. The higher the structure contribution, the higher the risk of damage to the critical structure from the reference beam.

In some embodiments, the structure contribution may be determined based on factors such as a dose of the reference beam deposited at the critical structure or a spatially informative relationship between the reference beam and the critical structure. For example, the higher the dose of the reference beam deposited in the critical structure, the higher the structure contribution. As another example, the shorter the distance between the center region of the irradiation range of the reference beam and the critical structure, the higher the structure contribution.

The target volume contribution refers to a metric used to measure a therapeutic benefit of a single reference beam to a single target volume. The higher the target volume contribution, the more significant the therapeutic benefit of the reference beam to the target volume.

In some embodiments, the target volume contribution may be determined based on a dose of the reference beam deposited at the target volume and the effect on characteristics such as dose uniformity and conformality of the target volume of the reference beam. For example, the processor may determine in real time the dose of the reference beam deposited on the target volume based on a Monte Carlo dose algorithm. The higher the dose, the more uniform the dose to the target volume, and the higher the conformality, the higher the target volume contribution. The conformality refers to a fit degree between the irradiation range of the reference beam and the range of the target volume. The larger the overlap area between the irradiation range of the reference beam and the range of the target volume, the better the fit degree, and the higher the conformality.

The importance factor refers to a metric used to measure the importance of a reference beam.

In some embodiments, the processor may determine the importance factor of the reference beam in a plurality of ways based on the structure contribution and the target volume contribution of the reference beam. For example, the processor may utilize a machine learning approach to obtain the importance factor corresponding to the reference beam. Exemplarily, for each reference beam, the processor may input the structure contribution corresponding to the critical structure, and the target volume contribution corresponding to the target volume into a factor prediction model to obtain the corresponding importance factor.

The factor prediction model may be a machine learning model, e.g., neural network (NN), etc.

In some embodiments, the factor prediction model may be obtained by training a plurality of first training samples with a first label. The first training samples are a sample structure contribution and a sample target volume contribution of a sample beam, the first label is an importance factor corresponding to the sample beam. The first training sample may be obtained based on experimental data or historical data, and the first label may be determined by manual labeling based on historical data or experimental results.

In some embodiments, the processor may input the plurality of first training samples with the first label into an initial factor prediction model, construct a loss function through the first label and the output of the initial factor prediction model, and iteratively update parameters of the initial factor prediction model based on the loss function through a gradient descent algorithm, etc. The training is completed when predetermined conditions are satisfied, and a trained factor prediction model is obtained. The predetermined conditions may be that the loss function converges, the count of iterations reaches a threshold, etc.

In some embodiments of the present disclosure, the structure contribution and the target volume contribution corresponding to each reference beam are designated as inputs of the factor prediction model. The factor prediction model processes and analyzes the input structure contribution and the target volume contribution to predict a suitable importance factor. By using the trained factor prediction model, the suitable importance factor for each reference beam is automatically predicted based on the input structure contribution and the target volume contribution, which is more efficient and accurate.

In some embodiments, for a reference beam, the processor may determine a first contribution based on the structure contribution of the reference beam corresponding to the critical structure, and the first contribution reflects the damage risk of the reference beam to the critical structure. The processor may also determine a second contribution based on the target volume contribution of the reference beam corresponding to the target volume, the second contribution reflecting the therapeutic benefit of the reference beam to the target volume. The processor may further determine the importance factor of the reference beam based on a relationship where the importance factor is negatively correlated with the first contribution and the importance factor is positively correlated with the second contribution.

The first contribution refers to a metric used to measure a total risk of injury to all critical structures from a single reference beam.

In some embodiments, the processor may perform a weighted summation of each structure contribution of the reference beam corresponding to each critical structure and determine the weighted summation as the first contribution. The weights may be generated based on clinical experience or an automated optimization algorithm. For example, in clinical experience, the importance of spinal cord is greater than the importance of muscle, then the weight of spinal cord is greater than the weight of muscle.

The second contribution refers to a metric used to measure a total therapeutic benefit of a single reference beam across all target volumes.

In some embodiments, the processor may sum the target volume contribution of the reference beam corresponding to each target volume and determine the weighted summation as the second contribution.

In some embodiments, the processor may determine the importance factor based on the first contribution and the second contribution. The importance factor is negatively correlated to the first contribution and positively correlated to the second contribution. For example, the importance factor corresponding to the reference beam may be determined based on the following equation (1):

$${IF}_{kj} = \frac{{\sum }_{m} ( {Contri}_{Target} )}{{\sum }_{n} ( {Contri}_{OAR} \cdot {W}_{OAR} )} . \qquad ( 1 )$$

In equation (1), IFₖⱼ denotes an importance factor of a j-th reference beam in the reference beam set corresponding to the k-th scanning point, Contri_{Target} denotes a target volume contribution of the j-th reference beam corresponding to an m-th target volume Target, Contri_{OAR} denotes a structure contribution of the j-th reference beam corresponding to an n-th critical structure OAR, W_{OAR} denotes a weight of the n-th critical structure OAR, Σₙ(Contri_{OAR}·W_{OAR}) denotes the first contribution, and Σₘ(Contri_{Target}) denotes the second contribution.

The weight corresponding to the critical structure OAR may be preset manually based on clinical experience.

By considering the first contribution and the second contribution while balancing the total therapeutic benefit of the reference beam across all target volumes against the total risk of injury to all critical structures, this approach overcomes the limitations of conventional single-target-single-organ optimization, thereby enabling quantitative assessment of the global utility of a single reference beam through the importance factor.

In 230, obtaining a plurality of target beam sets corresponding to the plurality of scanning points based on a plurality of importance factors corresponding to the plurality of reference beams, the plurality of reference beam sets, and a complexity control parameter.

The complexity control parameter refers to a metric used to measure the scanning spot beam loading. In some embodiments, the complexity control parameter may be a maximum count of target beams in a target beam set assigned to each scanning point.

In some embodiments, the larger the complexity control parameter, the greater the count of target beams in the target beam set in the arc radiotherapy plan, and the greater the uniformity of dose coverage in the target volume, but the slower the calculation of the processor. On the contrary, the smaller the complexity control parameter, the smaller the count of target beams in the target beam set in the arc radiotherapy plan, the lower the uniformity of dose coverage in the target volume, but the faster the calculation of the processor. A suitable complexity control parameter gets both the fastest calculation speed and the best plan quality.

The target beams refer to beams finalized for use in performing radiotherapy. The target beam set refers to a set that includes a plurality of target beams, the target beam set being a subset of the reference beam set. In some embodiments, one scanning point corresponds to one target beam set.

In some embodiments, the processor may obtain the plurality of target beam sets corresponding to the plurality of scanning points based on the plurality of importance factors corresponding to the plurality of reference beams, the plurality of reference beam sets, and the complexity control parameter in a plurality of ways. For example, the processor may combine the plurality of importance factors corresponding to the plurality of reference beams, the plurality of reference beam sets, and the complexity control parameter into an input vector. The input vector is input into a target beam set prediction model, and the target beam set is output.

The target beam set prediction model may be a machine learning model, e.g., neural network (NN), etc.

In some embodiments, the target beam set prediction model may be obtained by training a plurality of second training samples with a second label. The second training samples are a plurality of importance factors corresponding to a plurality of sample reference beams, the plurality of sample reference beam sets, and a sample complexity control parameter, and the second label is the target beam sets. The second training samples may be obtained based on experimental data or historical data, and the second label may be determined by manually labeling based on historical data or experimental results. The training process for the target beam set prediction model is similar to that of the factor prediction model, and may be referred hereinabove.

In some embodiments, the processor may also construct a particle source selection function based on the plurality of importance factors corresponding to the plurality of reference beams, the plurality of reference beam sets, and the complexity control parameter and perform an optimization solving operation on the particle source selection function to obtain the plurality of target beam sets corresponding to the plurality of scanning points.

In some embodiments, the particle source selection function may be represented by the following equation (2):

$$obj = {\sum }_{k} {\sum }_{j} ( {IF}_{kj} \cdot {x}_{kj} ) + {\sum }_{k} {\sum }_{j} ( θ \cdot card ( {J}_{k} \cdot {x}_{kj} ) ) , \qquad ( 2 )$$

s.t.

$${\sum }_{j} {x}_{kj} = min ( C , card ( {J}_{k} ) ) , j ∈ {J}_{k} ; {x}_{kj} ∈ \{ 0 , 1 \} ; C ∈ {I}^{+}$$

In the equation (2), obj denotes the particle source selection function, IFₖⱼ denotes the importance factor of the j-th reference beam in the reference beam set Jₖ corresponding to the k-th scanning point in the target volume, xₖⱼ denotes a selection variable, i.e., whether the k-th scanning point selects the j-th reference beam in the reference beam set Jₖ, xₖⱼ∈{0, 1}, xₖⱼ=0 denotes that the k-th scanning point does not select the j-th reference beam in the reference beam set Jₖ, xₖⱼ=1 denotes that the k-th scanning point selects the j-th reference beam in the reference beam set Jₖ, θ denotes a weighting factor of assigned balance, card denotes the count of sets, reference beams in the reference beam set, card(Jₖ) denotes the count of reference beams in the reference beam set Jₖ, card (Jₖ·xₖⱼ) denotes a count of selected reference beams in the reference beam set Jₖ, i.e., a count of selected beams corresponding to the k-th scanning point, C denotes the complexity control parameter, and I⁺ denotes a set of positive integers. Σₖ Σⱼ(IFₖⱼ·xₖⱼ) denotes the sum of the importance factors of all reference beams selected at all scanning points, and Σₖ Σⱼ(θ·card(Jₖ·xₖⱼ) denotes a weighted sum of the count of selected beams at each scanning point.

The weighting factor of assigned balance θ is used to balance the count of selected beams at each scanning point, and by posing a paradigm constraint on the count of selected beams at each scanning point by θ, the count of selected beams at each scanning point is more balanced.

Σⱼxₖⱼ=min(C,card(Jₖ), j∈Jₖ; xₖⱼ∈{0, 1}; C∈I⁺ is the constraint condition, representing the count of selected beams at the k-th scanning point is the minimum between the complexity control parameter and the count of reference beams in the reference beam set Jₖ corresponding to the k-th scanning point, the selection variable is a binary variable, and the complexity control parameter is a positive integer.

In some embodiments, the particle source selection function further includes an energy selector option characterizing the count of energy layers selected.

The energy layer refers to a beam level corresponding to the energy value output from the particle accelerator.

In some embodiments, the particle source selection function may also be represented by the following equation (3):

$$obj = {\sum }_{k} {\sum }_{j} ( {IF}_{kj} \cdot {x}_{kj} ) + {\sum }_{k} {\sum }_{j} ( θ \cdot card ( {J}_{k} \cdot {x}_{kj} ) ) + optional energy selector , \qquad ( 3 )$$

s.t.

$${\sum }_{j} {x}_{kj} = min ( C , card ( {J}_{k} ) ) , j ∈ {J}_{k} ; {x}_{kj} ∈ \{ 0 , 1 \} ; C ∈ {I}^{+} ,$$

where optional energy selector denotes the energy selector option, and more descriptions regarding obj, IFₖⱼ, xₖⱼ, θ, Jₖ, xₖⱼ, C, I⁺, card(Jₖ), Σₖ Σⱼ(IFₖⱼ·xₖⱼ), and Σₖ Σⱼ(θ·card(Jₖ·xₖⱼ)) can be found in the related descriptions in equation (2).

In some embodiments of the present disclosure, adding the energy selector option allows for optimized control to regulate the overall count of energy layers, and by assigning the energy selector option to the same energy as much as possible, the delivery time of the radiotherapy plan in the actual clinic is reduced, thereby increasing the delivery efficiency. The traditional method for generating the arc radiotherapy plan uses a stochastic greedy algorithm for screening the energy layers, and the radiotherapy plan obtained may be only locally optimal, with an unstable effect whereas some embodiments in the present disclosure allocate beams at the scanning point level rather than the energy layer, resulting in a stable and reproducible arc radiotherapy plan.

In some embodiments, the processor may solve the particle source selection function, such as by Benders decomposition to obtain the plurality of target beam sets corresponding to the plurality of scanning points.

In some embodiments, the processor may also obtain a plurality of priorities corresponding to a plurality of selection items in the particle source selection function and perform the optimization solving operation on the particle source selection function step-by-step based on the plurality of priorities to obtain the plurality of target beam sets corresponding to the plurality of scanning points.

In some embodiments, a selection term is an option that is evaluated to filter the underlying solution in the process for solving the particle source selection function.

In some embodiments, in conjunction with equation (2), the selection term may include a first selection term Σₖ Σⱼ(IFₖⱼ·xₖⱼ) determined based on the importance factor and a second selection term Σₖ Σⱼ(θ·card(Jₖ· xₖⱼ)) determined based on each reference beam set.

In some embodiments, in conjunction with equation (3), the selection item also includes the energy selector option optional energy selector.

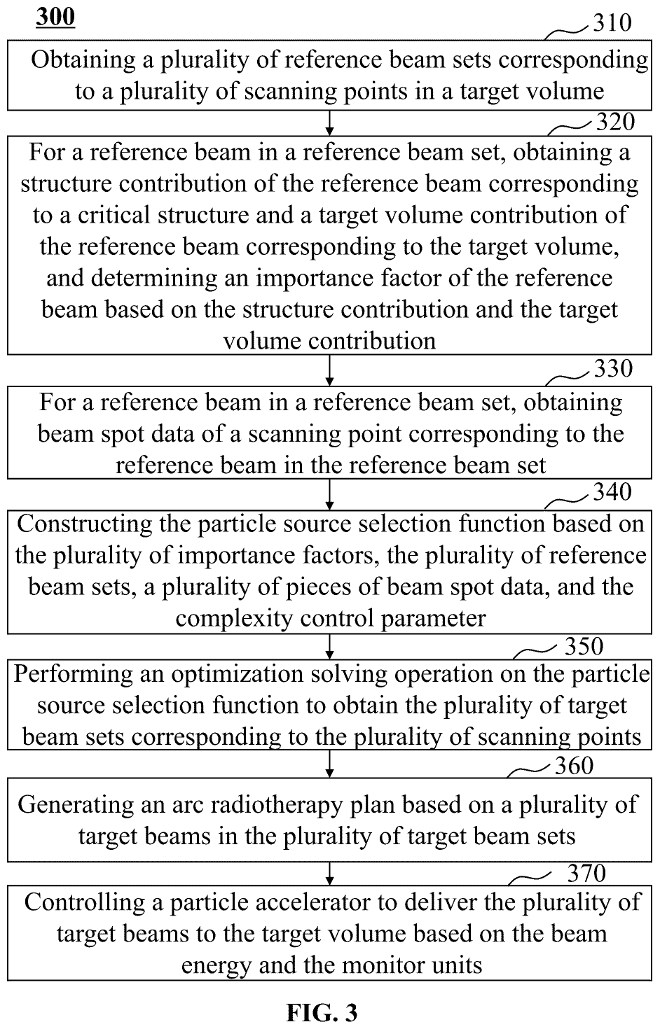

In some embodiments, in conjunction with equations (4) and (5) below, the selection term also includes a third selection term Σₖ Σⱼ(λ·σₖⱼ·xₖⱼ) determined based on beam spot data. Descriptions regarding the third option may be found in FIG. 3.

In some embodiments, the plurality of priorities corresponding to the plurality of selection items in the particle source selection function may be preset by the user based on clinical experience in conjunction with the actual needs. The priorities may be weight factors of the selection items.

In some embodiments, the processor may model the optimization problem with a plurality of scanning points as a spatially distributed network of decision variables, with each scanning point corresponding to a localized optimization problem and solve the problem under a constraint condition for a plurality of iterations to generate a globally optimal solution to determine the target beam sets.

In some embodiments of the present disclosure, the optimization problem of the particle source selection function is an integer optimization problem. However, in combination with the dose optimization problem, the beam intensity is introduced, making it a mixed integer planning problem. The traditional solution is time-consuming and affects the overall algorithmic efficiency. The embodiment of the present disclosure generates a final spot-scanning reassignment arc (SRArc) plan by setting the priority of solving each item in the optimization problem for step-by-step optimization and solving, to achieve the purpose of quickly solving the optimization problem and improving the overall computational efficiency.

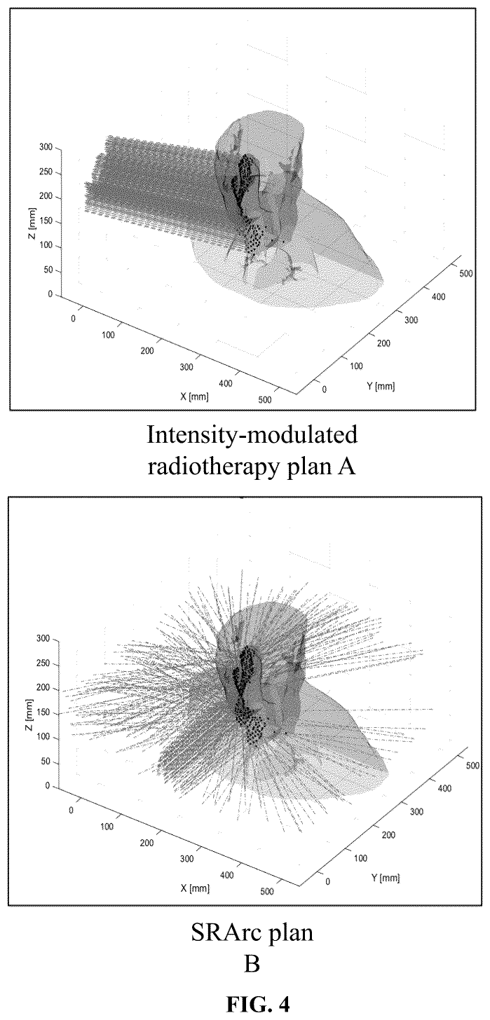

FIG. 4 is a schematic diagram illustrating a comparison between beam layouts according to some embodiments of the present disclosure.

Diagram A is a schematic diagram illustrating a distribution of beams of an intensity-modulated radiotherapy plan, representing the distribution of beams of the scanning points with the same energy layer in one field of the intensity-modulated radiotherapy plan. Diagram B is a schematic diagram illustrating a distribution of beams of the SRArc plan, representing the distribution of beams after the reassignment of beams of scanning points with the same energy layer in the SRArc plan.

In some embodiments of the present disclosure, the computational complexity and physical difficulty of treatment implementation are significantly reduced by uniformly optimizing the beam importance with the planning complexity, while ensuring dosage accuracy.

In 240, an arc radiotherapy plan is generated based on a plurality of target beams in the plurality of target beam sets.

The arc radiotherapy plan refers to a radiotherapy regimen formed based on the delivery of dose in a continuous arcuate path. In some embodiments, the arc radiotherapy plan includes beam energy and monitor units at a plurality of control points within an arc angle range. The arc angle range refers to a range of angles that the snout rotates.

The beam energy is the kinetic energy of the particle beam produced by the particle accelerator.

The monitor units serve as the beam intensity unit of the particle accelerator at the control points, which are used to regulate the output dose.

In some embodiments, the processor may generate the monitor units and beam energy of the control points based on the plurality of target beams in the plurality of target beam sets, by an inverse optimization algorithm, such as sequential quadratic programming (sequential quadratic programming (SQP), a limited-memory Broyden-Fletcher-Goldfarb-Shanno algorithm (L-BFGS), or an interior-point manner).

In some embodiments, the processor may obtain a plurality of pieces of dose distribution data corresponding to the plurality of target beams in the plurality of target beam sets, determine a dose deposition matrix based on the plurality of pieces of dose distribution data, and determine a plurality of monitor units corresponding to the plurality of target beams in the arc radiotherapy plan based on the dose deposition matrix, a tissue weighting factor and a dose threshold corresponding to the critical structure.

The dose distribution data refers to three-dimensional distribution data of the dose deposited by the target beam within the target object. In some embodiments, the processor may calculate the dose distribution data by Monte Carlo simulation for the beam energy, rack angle, MLC shape, treatment bed position, or the like.

In some embodiments, the processor may obtain the dose distribution data based on the target beam via Monte Carlo simulation, a penumbral beam convolution algorithm, or the like. For example, the target beams (including a rack angle, MLC shape, energy, etc.) are input into a Monte Carlo simulation engine to obtain the corresponding dose distribution data.

The dose deposition matrix refers to a matrix expression of the dose distribution data of the plurality of target beams to a plurality of voxels in the target object, with voxels in the target object as the rows, and the target beams as the columns, and each element within the matrix represents the dose of one target beam to a voxel.

The dose deposition matrix is used to characterize the dose delivered to the voxel by the target beam per unit of luminous flux.

In some embodiments, the processor may generate the dose deposition matrix based on the plurality of pieces of dose distribution data using, e.g., Monte Carlo simulation, by calculation. For example, the plurality of pieces of dose distribution data are input into the Monte Carlo simulation engine to obtain a corresponding dose deposition matrix.

The dose threshold refers to a safe threshold for the deposition of dose in the critical structure, which may be preset manually based on clinical experience.

In some embodiments, the processor may select a relative biological effect model, set a constraint condition such as the tissue weighting factor and the dose threshold corresponding to the critical structure, construct a dose distribution optimization function for optimization calculation, and solve in an optimization solver to obtain the monitor units corresponding to each target beam in the arc radiotherapy plan. Exemplarily, the optimization solver may be a large-scale, high-dimensional, nonlinear optimization solver, such as interior point optimizer (IPOPT), and the optimization solver is not limited herein. In some embodiments, the optimization algorithm used for the dose distribution optimization function is a robust optimization algorithm or a bio-effect optimization algorithm.

Conventional multi-angle intensity-modulated radiotherapy plan for a given range requires a plurality of iterations and interactions, with each iteration requiring the computation of the dose deposition matrix. Depending on the physical nature of the particle beam stream, each reference beam affects a plurality of voxels both laterally and axially, which follow a certain three-dimensional spatial distribution. However, the dose deposition matrix and the dose distribution optimization algorithm consume substantial computing power, and repeated calculations of different combinations of dose deposition matrices waste significant computation time, which reduces the efficiency of the algorithm and does not have practical clinical value for large-scale cases. In some embodiments of the present disclosure, the particle source selection function may calculate a target beam set with controlled complexity at one time, and the dose deposition matrix only needs to be calculated once during the entire calculation of the radiotherapy plan, thereby saving the time required to generate the radiotherapy plan.

In 250, the particle accelerator is controlled to deliver the plurality of target beams to the target volume based on the beam energy and the monitor units.

In some embodiments, each control point corresponds to a specific rack angle, and the particle accelerator adjusts the shape of the leaves of a multi-leaf collimator (MLC) in real time to match the tumor contour according to the arc radiotherapy plan, and synchronously controls the dose rate and the allocation of the monitor units to ensure that the energy and dose of the beams at different angles are accumulated according to the arc radiotherapy plan. With the continuous rotation of the rack, these dynamic parameters are smoothly transitioned between the control points, so that the multi-angle beams may focus on the target volume from an arc path, ultimately achieving high-precision treatment on the targets through dose superposition, while maximizing the protection of surrounding normal tissues.

In some embodiments of the present disclosure, by determining the importance factor corresponding to each reference beam based on the structure contribution corresponding to the critical structure and the target volume contribution corresponding to the target volume of each reference beam in each reference beam set, constructing the particle source selection function based on each importance factor, each reference beam set, and the complexity control parameter, performing the optimization solving operation on the particle source selection function to obtain the target beam set corresponding to each scanning point, and generating the arc radiotherapy plan based on each target beam in each target beam set, the problem of the poor quality of the relevant radiotherapy plan is solved. In addition, under the prerequisite of guaranteeing that the target volume is completely covered, the dose to the critical structures is reduced as much as possible from the perspective of beam quantization, the advantages of arc scanning are fully utilized, the errors caused by manually selecting the angle of the field are avoided, and the dose conformity of the target volume is improved. Moreover, the embodiment of the present disclosure assigns the beams at the scanning point level rather than the energy layer, so as to make the result of the generated arc radiotherapy plan stable and repeatable. At the same time, the degree of freedom of the optimization problem is improved, allowing the solution space of the optimization problem to be expanded so that higher quality arc radiotherapy plans may be obtained.

FIG. 3 is a flowchart illustrating a process for generating an arc radiotherapy plan according to some other embodiments of the present disclosure.

As shown in FIG. 3, process 300 includes the following operations. In some embodiments, the process 300 may be executed by a processor.

In 310, a plurality of reference beam sets corresponding to a plurality of scanning points in a target volume are obtained. More descriptions regarding operation 310 may be found in the related description of operation 210 in FIG. 2.

In 320, for a reference beam in a reference beam set, a structure contribution of the reference beam corresponding to a critical structure and a target volume contribution of the reference beam corresponding to the target volume are obtained, and an importance factor of the reference beam is determined based on the structure contribution and the target volume contribution. More descriptions regarding operation 320 may be found in the related description of operation 220 in FIG. 2.

In 330, for a reference beam of a reference beam set, beam spot data of a scanning point corresponding to the reference beam in the reference beam set is obtained.

The beam spot data refers to a parameter describing the localized dose distribution characteristics of a beam at a scanning point within a target object, including a transverse beam spot dimension, longitudinal dose attenuation, an off-axis ratio, or the like. In some embodiments, the beam spot data may be used to characterize the beam spot size at which the reference beam arrives at the scanning point, with the beam spot data being determined by parameters such as the energy layer, a source model, a virtual source-axis distance (VSAD), a snout position, or the like. The beam characteristics include a beam energy, a dose rate, a source dimension, or the like.

In some embodiments, the processor may obtain beam spot data based on the beam characteristics calibrated by the treatment machine by calculating through a Monte Carlo simulation or a penumbral beam algorithm.

In 340, a particle source selection function is constructed based on a plurality of importance factors, a plurality of reference beam sets, a plurality of pieces of beam spot data, and a complexity control parameter.

In some embodiments, the particle source selection function reflects the combined influence of three parts.

The first part uses the importance factor as a treatment utility indicator, reflecting a total therapeutic utility of selected reference beams, and the first part is configured to prioritize a high-efficacy beam. The high-efficacy beam refers to a reference beam that delivers a higher dose to the target volume with less impact on the critical structure during radiotherapy.

The second part uses the beam spot data as a precision error indicator, reflecting a total precision error of the selected reference beams, and the second portion is configured to reduce a selection probability of beams with large spot sizes. The beams with large spot sizes refer to reference beams with large spot sizes at scanning points within the target object.

The third part uses a count of beams as an allocation balance indicator, reflecting an allocation balance of the selected reference beams, and the third part is configured to limit beam overload in a local region.

In some embodiments, the constraint condition includes that: for a scanning point, a count of selected beams of the scanning point is determined based on the complexity control parameter and the count of reference beams in a reference beam sets at the scanning point, the selection variable is binary, and the complexity control parameter is a positive integer. The constraint condition is used to constrain the count of selected beams at each scanning point. For example, the count of selected beams at the scanning point is the minimum of the complexity control parameter and the count of reference beams of the reference beam set corresponding to that scanning point.

In some embodiments, the particle source selection function may be expressed based on the first part, the second part, and the third part by the following equation (4):

$$obj = {\sum }_{k} {\sum }_{j} ( {IF}_{kj} \cdot {x}_{kj} ) + {\sum }_{k} {\sum }_{j} ( λ \cdot {σ}_{kj} \cdot {x}_{kj} ) + {\sum }_{k} {\sum }_{j} ( θ \cdot card ( {J}_{k} \cdot {x}_{kj} ) ) , \qquad ( 4 )$$

s.t.

$${\sum }_{j} {x}_{kj} = min ( C , card ( {J}_{k} ) ) , j ∈ {J}_{k} ; {x}_{kj} ∈ \{ 0 , 1 \} ; C ∈ {I}^{+}$$

In equation (4), A denotes the weight parameter of the beam spot data, and σₖⱼ denotes the beam spot data of the j-th reference beam arriving at the k-th scanning point in the target volume. Σₖ σⱼ(λ·σₖⱼ·xₖⱼ) denotes the weighted sum of beam spot data for all reference beams selected at all scanning points. Other parameter explanations may be found in the description of equations (1)-(3).

The weighting parameter of the beam spot data A is used to indicate the relative degree of contribution of the beam spot data to the dose distribution. By adjusting the weights of different beam spot data, the dose contribution of each voxel is controlled so that the target volume achieves a uniformly high dose while avoiding the critical structure. The weighting parameter of the beam spot data may be preset manually based on clinical experience.

Σₖ Σⱼ(IFₖⱼ·xₖⱼ) denotes the first part, Σₖ Σⱼ(λ·σₖⱼ·xₖⱼ) denotes the second part, and Σₖ Σⱼ(θ·card(Jₖ·xₖⱼ)) denotes the third part.

In some embodiments of the present disclosure, by constructing the particle source selection function including three parts, it is possible to comprehensively consider a variety of factors, such as the importance factor of the beam, the beam spot data, and the weighting factor of assigned balance, and to achieve a comprehensive evaluation and optimization of the beams. The first part of the particle source selection function selects the local high-efficiency beam based on the importance factor, which helps to improve the treatment effect and the accuracy of the dose coverage in the target volume. The second part suppresses the error data based on the beam spot data, which effectively reduces the uneven dose distribution and improves the accuracy of treatment. The third part balances the count of beams based on the weighting factor of assigned balance, ensuring that the beams at each scanning point are reasonably distributed, and avoiding the dose deviation caused by the uneven distribution of beams. In addition, the constraint condition rationally determines the count of selected beams by controlling the complexity parameter, which ensures the feasibility and operability of the treatment plan, and at the same time avoids the problems caused by the excessive or insufficient count of beams, thus enhancing the quality and safety of the whole radiotherapy plan.

In some embodiments, the particle source selection function may also be represented by the following equation (5):

$$obj = {\sum }_{k} {\sum }_{j} ( {IF}_{kj} \cdot {x}_{kj} ) + {\sum }_{k} {\sum }_{j} ( λ \cdot {σ}_{kj} \cdot {x}_{kj} ) + {\sum }_{k} {\sum }_{j} ( θ \cdot card ( {J}_{k} \cdot {x}_{kj} ) ) + optional energy selector , \qquad ( 5 )$$

s.t.

$${\sum }_{j} {x}_{kj} = min ( C , card ( {J}_{k} ) ) , j ∈ {J}_{k} ; {x}_{kj} ∈ \{ 0 , 1 \} ; C ∈ {I}^{+}$$

In equation (5), optional energy selector denotes the energy selector option, and the other parameters may be found in the description of equations (2)-(4).

In some embodiments of the present disclosure, the particle source selection function is constructed by combining the beam spot data of the reference beam and the importance factor, or the like, from which a fine control of the beam selection is realized, to make the particle source selection function more closely fit the actual radiotherapy needs. The acquisition of the beam spot data helps to evaluate the dose distribution characteristics of the reference beam at the scanning point, so that the particle source selection function may more accurately reflect the contribution of the beams to the target volume and the potential impact on the surrounding normal tissues. Meanwhile, by combining the importance factor and information from a plurality of reference beam sets, the particle source selection function may synthesize the properties of each beam and thus select the optimal beam combination. Additionally, the introduction of the complexity control parameter helps to control the complexity of the plan while ensuring the quality of the radiotherapy plan, reducing the difficulty of calculation and implementation.

In 350, an optimization solving operation is performed on the particle source selection function to obtain the plurality of target beam sets corresponding to the plurality of scanning points.

In some embodiments, as described in relation to operation 230 in FIG. 2, the processor may obtain a plurality of priorities corresponding to a plurality of selection items in the particle source selection function, and perform the optimization solving operation on the particle source selection function step-by-step based on the plurality of priorities to obtain the plurality of target beam sets corresponding to the plurality of scanning points. For Equation (4), the selection items may include a first selection item, a second selection item, and a third selection item; for equation (5), the selection items may include a first selection item, a second selection item, a third selection item, and an energy selector option.

In 360, the arc radiotherapy plan is generated based on a plurality of target beams in the plurality of target beam sets. More descriptions regarding operation 360 may be found in the related description of operation 240 in FIG. 2.

In 370, the particle accelerator is controlled to deliver the plurality of target beams to the target volume based on the beam energy and the monitor units. More descriptions regarding operation 370 may be found in the related description of operation 250 in FIG. 2.

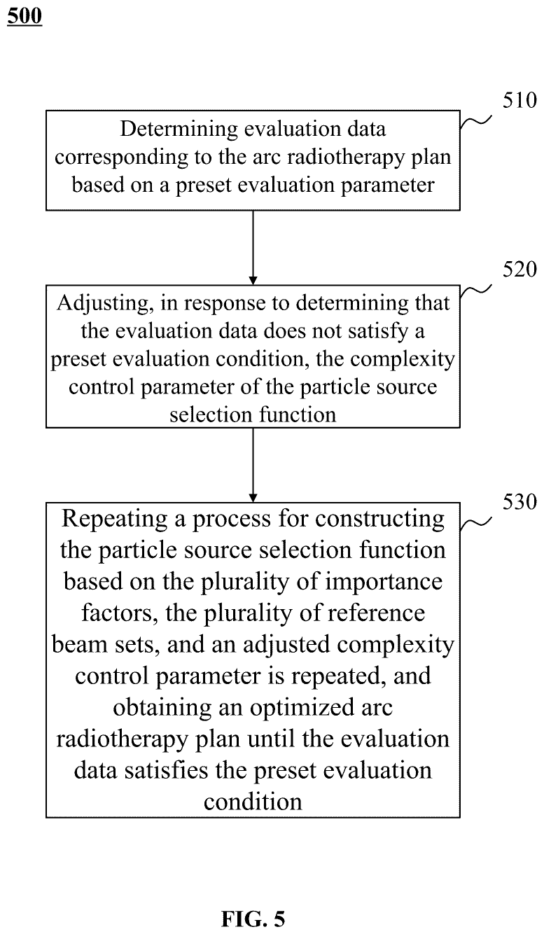

FIG. 5 is a flowchart illustrating a process for optimizing an arc radiotherapy plan according to some embodiments of the present disclosure. As shown in FIG. 5, process 500 includes the following operations. In some embodiments, the process 500 may be executed by a processor.

In 510, evaluation data corresponding to the arc radiotherapy plan is determined based on a preset evaluation parameter.

The preset evaluation parameter refers to a parameter set for evaluating the arc radiotherapy plan. The preset evaluation parameter includes but is not limited to, an isodose curve, a dose-volume histogram, a dose uniformity, etc.

In some embodiments, the preset evaluation parameter may be manually pre-set based on clinical experience and practical needs.

The evaluation data refers to a quantitative result of the preset evaluation parameter generated by dose calculations performed on the arc radiotherapy plan.

In some embodiments, the processor may generate the evaluation data corresponding to the arc radiotherapy plan based on the preset evaluation parameter via a dose calculation engine.

In 520, in response to determining that the evaluation data does not satisfy a preset evaluation condition, the complexity control parameter of the particle source selection function is adjusted.

The preset evaluation condition refers to a preset condition to be met by the evaluation data. For example, the preset evaluation condition may be that the isodose curve covers an area of the target volume that exceeds a preset area proportionality threshold, that the dose-volume histograms of all the critical structures do not exceed a preset volume-dose limit, and that the dose homogeneity of the target volume meets a preset range. In some embodiments, the preset evaluation condition may be manually predetermined based on clinical experience and practical needs.

In some embodiments, the processor may dynamically adjust the complexity control parameter in the particle source selection function in response to the results of comparing the evaluation data with the preset evaluation condition. For example, if the evaluation data of the isodose curve does not satisfy the corresponding preset evaluation condition, the value of the complexity control parameter is increased to allow more beams to participate in the optimization process.

In 530, a process for constructing the particle source selection function based on the plurality of importance factors, the plurality of reference beam sets, and an adjusted complexity control parameter is repeated, and an optimized arc radiotherapy plan is obtained until the evaluation data satisfies the preset evaluation condition.

In some embodiments, the processor first constructs the particle source selection function based on an initial complexity control parameter and solves to obtain a target beam set, which in turn generates an arc radiotherapy plan. The evaluation data of the arc radiotherapy plan is determined using the preset evaluation parameter and compared to the preset evaluation condition. If the evaluation data does not satisfy the preset evaluation condition, the complexity control parameter is adjusted, and the particle source selection function is constructed and solved again, then the process is repeated until the evaluation data meets the preset evaluation condition, and ultimately the optimized arc radiotherapy plan is obtained.

In some embodiments of the present disclosure, the adaptability of the treatment plan to the specific situation of the target object is enhanced by adjusting the complexity control parameter in the particle source selection function in the event that the evaluation data does not satisfy the preset evaluation condition. This optimization mechanism enables the arc radiotherapy plan to be adjusted based on real-time feedback, which helps to achieve a more accurate dose distribution while improving target volume coverage and enhancing treatment effectiveness.

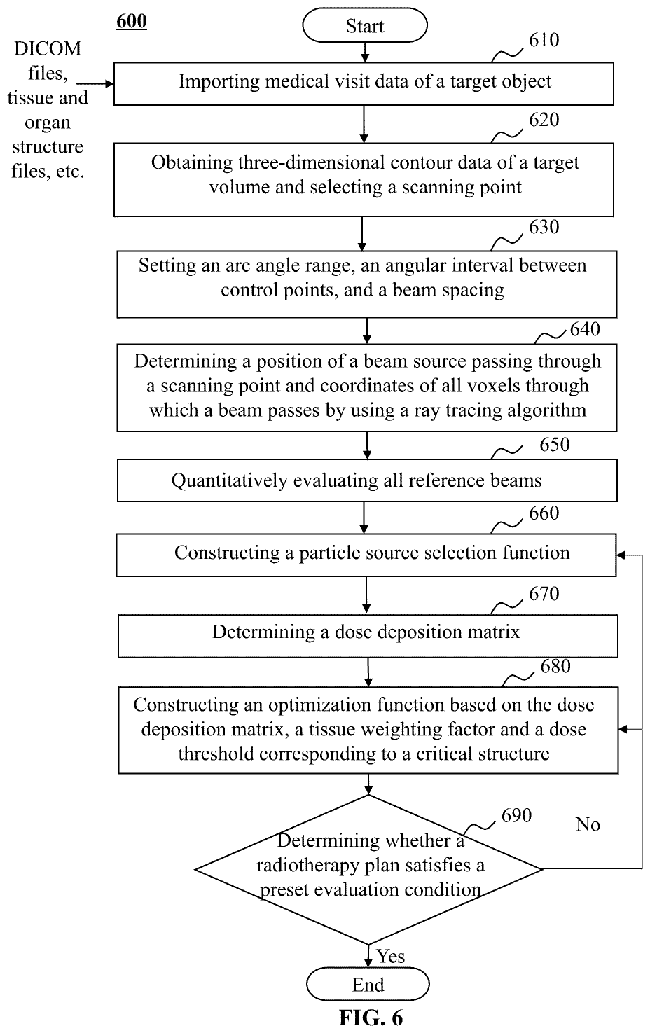

FIG. 6 is a flowchart illustrating an embodiment of a process for generating an arc radiotherapy plan according to some embodiments of the present disclosure. In some embodiments, process 600 includes the following operations, and the process 600 may be executed by a processor.

In 610, medical visit data of a target object is imported.

The medical visit data includes but is not limited to digital imaging and communications in medicine (DICOM) files, tissue and organ structure files, etc., and the DICOM files typically contain information such as tissue density values measured in Heinz units and the coordinate values of the image in the reference coordinate system of the body.

In some embodiments, the processor may outline a tissue and organ structure file in the medical visit data to obtain volume and three-dimensional contour data corresponding to each tissue or organ, respectively. Each tissue or organ includes at least one target volume and at least one critical structure.

In 620, three-dimensional contour data of a target volume is obtained, and a scanning point is selected.

Descriptions regarding this section may be found in operation 210 in FIG. 2.

In 630, an arc angle range, an angular interval between control points, and a beam spacing are set.

More about this section can be found in the FIG. 2 related description.

In 640, a position of the beam source passing through a scanning point and coordinates of all the voxels through which a beam passes are determined using a ray tracing algorithm.

Descriptions regarding this section may be found in operation 210 in FIG. 2.

In 650, all reference beams are quantitatively evaluated.

In some embodiments, the process for quantitatively evaluating a reference beam refers to a process for determining an importance factor and beam spot data corresponding to the reference beam. Descriptions regarding determining the importance factor and beam spot data may be found in the related descriptions of operation 220 in FIG. 2 and operations 320-330 in FIG. 3.

In 660, the particle source selection function is constructed.

Descriptions regarding this section may be found in operation 230 in FIG. 2 and operation 340 in FIG. 3.

In 670, a dose deposition matrix is determined.

In some embodiments, the processor may determine the dose deposition matrix based on a plurality of pieces of dose distribution data.

Descriptions regarding this section may be found in operation 240 in FIG. 2.

In 680, an optimization function is constructed based on the dose deposition matrix, and a tissue weighting factor and a dose threshold corresponding to a critical structure.

In some embodiments, the processor may construct a dose distribution optimization function based on the dose deposition matrix, the tissue weighting factor and the dose threshold corresponding to the critical structure, and solve the function in an optimization solver to obtain an optimization result, which is monitor units corresponding to each target beam in the arc radiotherapy plan. More descriptions regarding this section may be found in the description of FIG. 2.

In 690, whether the evaluation data of the arc radiotherapy plan obtained based on the optimization result satisfies a preset evaluation condition is determined.

If the evaluation data satisfies the preset evaluation condition, the process is ended and the arc radiotherapy plan is obtained. If the evaluation data does not satisfy the preset evaluation condition, the complexity control parameter in the particle source selection function is adjusted, the process for constructing the particle source selection function based on each importance factor, each reference beam set, and the complexity control parameter is repeated based on the adjusted complexity control parameter, and/or, the tissue weighting factor and the dose threshold corresponding to the critical structure in the optimization function are adjusted, or the relative biological effect model selected in the optimization function is adjusted, and the process for constructing the optimization function based on the dose deposition matrix, the tissue weighting factor and the dose threshold corresponding to the critical structure is repeated.

In some embodiments of the present disclosure, by obtaining, for each reference beam set, the beam spot data of each reference beam in the reference beam set corresponding to the scanning point, and constructing the particle source selection function based on each importance factor, each reference beam set, each piece of beam spot data, and the complexity control parameter, the particle source selection function is further improved, and the constraint optimization of the target beam set is performed from the beam spot perspective of the beams, which further improves the quality of the arc radiotherapy plan.

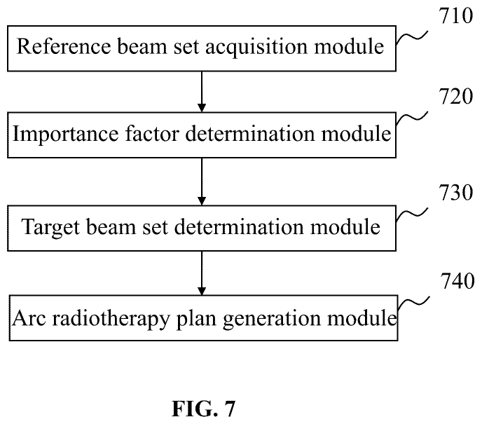

FIG. 7 is a schematic diagram illustrating an apparatus for generating an arc radiotherapy plan according to some embodiments of the present disclosure. As shown in FIG. 7, the apparatus includes a reference beam set acquisition module 710, an importance factor determination module 720, a target beam set determination module 730, and an arc radiotherapy plan generation module 740. The specific implementations thereof are consistent with the implementations documented in the above-described embodiments of the method, the technical effects achieved, and some of which will not be repeated.

The reference beam set acquisition module 710 is configured to obtain a plurality of reference beam sets corresponding to a plurality of scanning points in the target volume, and for a reference beam in a reference beam set, to obtain a structure contribution of the reference beam corresponding to a critical structure and a target volume contribution of the reference beam corresponding to the target volume.

The importance factor determination module 720 is configured to determine an importance factor corresponding to a reference beam based on the structure contribution and the target area contribution.

The target beam set determination module 730 is configured to obtain a plurality of target beam sets corresponding to a plurality of scanning points based on a plurality of importance factors corresponding to a plurality of reference beams, the plurality of reference beam sets, and a complexity control parameter.

The arc radiotherapy plan generation module 740 is configured to generate an arc radiotherapy plan based on a plurality of target beams in the plurality of target beam sets, and the arc radiotherapy plan includes beam energy and monitor units for a plurality of control points within an arc angle range.

In some embodiments, the apparatus for generating the arc radiotherapy plan may further include a control module, which may be configured to control a particle accelerator to deliver a plurality of target beams to the target volume based on the beam energy and the monitor units.

In some embodiments, the reference beam set acquisition module 710, the importance factor determination module 720, the target beam set determination module 730, and the arc radiotherapy plan generation module 740 may be integrated in the processor 110, and the control module may be configured in the processor 110, the terminal device 140, and/or the treatment machine 150.

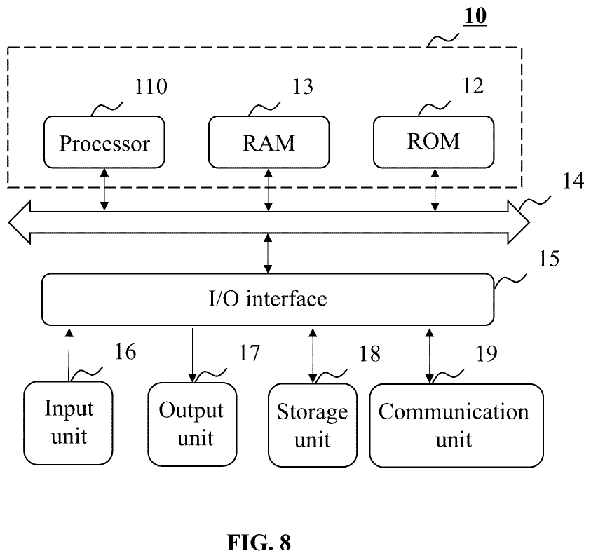

FIG. 8 is a schematic diagram illustrating an electronic device according to some embodiments of the present disclosure. An electronic device 10 is intended to represent various forms of digital computers, such as laptops, desktop computers, workstations, personal digital assistants, servers, blade servers, mainframe computers, and other suitable computers. The electronic device 10 may also represent various forms of mobile devices such as personal digital processing, cellular telephones, smartphones, wearable devices (e.g., helmets, eyeglasses, watches, etc.), and other similar computing devices. Connections and relationships, as well as the functionality of the components shown in the embodiments of the present disclosure, are shown as examples only and are not intended to limit the implementations of the present disclosure described and/or claimed herein.

As shown in FIG. 8, the electronic device 10 includes at least one processor 110, and memory, such as read-only memory (ROM) 12, random access memory (RAM) 13, or the like, communicatively connected to the at least one processor 110. The memory stores computer programs executable by the at least one processor 110, and the processor 110 may perform various appropriate actions and processes based on the computer programs stored in the ROM 12 or a computer program loaded from a storage unit 18 into the RAM 13. In the RAM 13, various programs and data required for the operation of the electronic device 10 may also be stored. The processor 110, the ROM 12, and the RAM 13 are connected to each other via a bus 14. An input/output (I/O) interface 15 is also connected to the bus 14.

A plurality of components in the electronic device 10 are connected to an I/O interface 15, including an input unit 16 (e.g., a keyboard, a mouse, or the like), an output unit 17 (e.g., various types of monitors, speakers, or the like), a storage unit 18 (e.g., a disk, a compact disk, or the like), and a communication unit 19 (e.g., a network card, a modem, a wireless communication transceiver, etc.). The communication unit 19 allows the electronic device 10 to exchange information/data with other devices via a computer network, such as the Internet, and/or various telecommunications networks.

Descriptions regarding the processor 110 may be found in the related description in FIG. 1.

In some embodiments, the method for generating the arc radiotherapy plan may be implemented as a computer program that is tangibly contained in a computer-readable storage medium such as the storage unit 18. In some embodiments, portions or all of the computer program may be loaded and/or mounted onto the electronic device 10 via the ROM 12 and/or the communication unit 19. When the computer program is loaded into the RAM 13 and executed by the processor 11, one or more of the operations of the method for generating the arc radiotherapy plan described above may be performed. Alternatively, in other embodiments, the processor 110 may be configured to perform the method for generating the arc radiotherapy plan by any other appropriate manners (e.g., with the aid of firmware).

Various embodiments of the systems and techniques described above herein may be realized in digital electronic circuit systems, integrated circuit systems, field programmable gate arrays (FPGAs), application-specific integrated circuits (ASICs), application-specific standard products (ASSPs), systems of systems-on-a-chip (SOCs), loaded programmable logic devices (CPLDs), computer hardware, firmware, software, and/or a combination thereof. These embodiments may include implementation in one or more computer programs that may be executed and/or interpreted on a programmable system including at least one programmable processor, which may be a dedicated or general-purpose programmable processor that may receive data and instructions from the storage system, the at least one input device, and the at least one output device, and transmit the data and instructions to the storage system, the at least one input device, and the at least one output device.

One embodiment of the present disclosure further provides a non-transitory computer-readable storage medium storing computer instructions. The computer instructions are used to cause the processor to perform the method for generating the arc radiotherapy plan. The method includes obtaining a plurality of reference beam sets corresponding to a plurality of scanning points in a target volume; for a reference beam in a reference beam set, obtaining a structure contribution of the reference beam corresponding to a critical structure and a target volume contribution of the reference beam corresponding to the target volume, and determining an importance factor of the reference beam based on the structure contribution and the target volume contribution; obtaining a plurality of target beam sets corresponding to the plurality of scanning points based on a plurality of importance factors corresponding to the plurality of reference beams, the plurality of reference beam sets, and a complexity control parameter; generating an arc radiotherapy plan based on a plurality of target beams in the plurality of target beam sets, where the arc radiotherapy plan includes beam energy and monitor units for a plurality of control points within an arc angle range; and controlling a particle accelerator to deliver the plurality of target beams to the target volume based on the beam energy and the monitor units.

In some embodiments, the computer-readable storage medium may be a tangible medium that may contain or store a computer program for use by, or in conjunction with, an instruction-execution system, device, or apparatus. The computer-readable storage medium may include but are not limited to electronic, magnetic, optical, electromagnetic, infrared, or semiconductor system, apparatus, or device, or any suitable combination of the foregoing. Alternatively, the computer-readable storage medium may be a machine-readable signaling medium. More specific embodiments of the machine-readable storage medium may include electrical connections based on one or more wires, portable computer disks, hard disks, RAM, ROM, erasable programmable read-only memory (EPROM or flash memory), optical fibers, convenient compact disk read-only memory (CD-ROM), optical storage devices, magnetic storage devices, or any suitable combination of the foregoing.

To provide interaction with a user, the systems and techniques described herein may be implemented on an electronic device including a display device (e.g., a cathode ray tube (CRT) or liquid crystal display (LCD) monitor) for displaying information to the user and a keyboard and a pointing device (e.g., a mouse or a trackball) by which the user may provide input to the electronic device. Other kinds of devices may also be configured to provide interaction with the user. For example, the feedback provided to the user may be any form of sensory feedback (e.g., visual feedback, auditory feedback, or haptic feedback). Input from the user may be received in any form (including acoustic input, voice input, or haptic input).

The systems and techniques described herein may be implemented in a computing system that includes a back-end component (e.g., as a data server), or a computing system that includes a middleware component (e.g., an application server), or a computing system that includes a front-end component (e.g., a user's computer that has a graphical user interface or a web browser through which the user may interact with the systems and techniques described herein), or a computing system that includes any combination of such back-end, middleware, or front-end components. The components of the system may be interconnected by digital data communication in any form or medium (e.g., a communications network). Embodiments of communication networks include local area networks (LANs), wide area networks (WANs), blockchain networks, and the Internet.

The computing system may include a client and a server. The client and the server are generally remote from each other and typically interact over a communication network. A client-server relationship is generated by computer programs that run on corresponding computers and have a client-server relationship with each other. The server may be a cloud server, also known as a cloud computing server or cloud host, which is a host product in the cloud computing service system to solve the existence of the difficulty of management, and the weakness of business scalability in the traditional physical hosts and VPS services.

In addition, certain features, structures, or characteristics of one or more embodiments of the present disclosure may be suitably combined.

In the event of any inconsistency or conflict between the descriptions, definitions, and/or use of terms in the materials cited in the present disclosure and those described in the present disclosure, the descriptions, definitions, and/or use of terms in the present disclosure shall prevail.

What is claimed is:

1. A method for generating an arc radiotherapy plan, comprising:
obtaining a plurality of reference beam sets corresponding to a plurality of scanning points in a target volume;
for a reference beam in one of the plurality of reference beam sets, obtaining a structure contribution of the reference beam corresponding to a critical structure and a target volume contribution of the reference beam corresponding to the target volume, and determining an importance factor of the reference beam based on the structure contribution and the target volume contribution;
obtaining a plurality of target beam sets corresponding to the plurality of scanning points based on a plurality of importance factors corresponding to a plurality of reference beams, the plurality of reference beam sets, and a complexity control parameter;
generating the arc radiotherapy plan based on a plurality of target beams in the plurality of target beam sets, wherein the arc radiotherapy plan includes beam energy and monitor units for a plurality of control points within an arc angle range; and
controlling a particle accelerator to deliver the plurality of target beams to the target volume based on the beam energy and the monitor units.

2. The method of claim 1, wherein the obtaining a plurality of target beam sets corresponding to the plurality of scanning points based on a plurality of importance factors corresponding to a plurality of reference beams, the plurality of reference beam sets, and a complexity control parameter includes:
constructing a particle source selection function based on the plurality of importance factors, the plurality of reference beam sets, and the complexity control parameter, and performing an optimization solving operation on the particle source selection function to obtain the plurality of target beam sets corresponding to the plurality of scanning points.

3. The method of claim 1, wherein the determining an importance factor of the reference beam based on the structure contribution and the target volume contribution includes:
for the reference beam,
determining a first contribution based on the structure contribution of the reference beam corresponding to the critical structure, wherein the first contribution reflects a damage risk of the reference beam to the critical structure;
determining a second contribution based on the target volume contribution of the reference beam corresponding to the target volume, wherein the second contribution reflects a therapeutic benefit of the reference beam to the target volume; and
determining the importance factor of the reference beam based on a relationship where the importance factor is negatively correlated with the first contribution and the importance factor is positively correlated with the second contribution.

4. The method of claim 2, wherein the constructing a particle source selection function based on the plurality of importance factors, the plurality of reference beam sets, and the complexity control parameter includes:
for a reference beam in one of the plurality of reference beam sets, obtaining beam spot data of a scanning point corresponding to the reference beam in the reference beam set; and
constructing the particle source selection function based on the plurality of importance factors, the plurality of reference beam sets, a plurality of pieces of beam spot data, and the complexity control parameter.

5. The method of claim 4, wherein the particle source selection function satisfies the following conditions:
the particle source selection function reflects a combined influence of three parts, and the three parts include a first part, a second part, and a third part, wherein:
the first part uses the importance factor as a treatment utility indicator, reflecting a total treatment utility of selected reference beams, and the first part is configured to prioritize a high-efficacy beam;
the second part uses the beam spot data as a precision error indicator, reflecting a total precision error of the selected reference beams, and the second part is configured to reduce a selection probability of beams with large spot sizes; and
the third part uses a count of beams as an allocation balance indicator, reflecting an allocation balance of the selected reference beams, and the third part is configured to limit beam overload in a local region; and
a constraint condition of the particle source selection function includes:
for a scanning point, a count of selected beams of the scanning point being determined based on the complexity control parameter and a count of reference beams in a reference beam set of the scanning point, wherein a selection variable is a binary variable, and the complexity control parameter is a positive integer.

6. The method of claim 5, further comprising:
determining evaluation data corresponding to the arc radiotherapy plan based on a preset evaluation parameter;
adjusting, in response to determining that the evaluation data does not satisfy a preset evaluation condition, the complexity control parameter of the particle source selection function; and
repeating a process for constructing the particle source selection function based on the plurality of importance factors, the plurality of reference beam sets, and an adjusted complexity control parameter, and obtaining an optimized arc radiotherapy plan until the evaluation data satisfies the preset evaluation condition.

7. The method of claim 2, wherein the particle source selection function further includes an energy selector option, and the energy selector option characterizes a count of selected energy layers.

8. The method of claim 2, wherein the performing an optimization solving operation on the particle source selection function to obtain the plurality of target beam sets corresponding to the plurality of scanning points includes:
obtaining a plurality of priorities corresponding to a plurality of selection items in the particle source selection function; and
performing the optimization solving operation on the particle source selection function step-by-step based on the plurality of priorities to obtain the plurality of target beam sets corresponding to the plurality of scanning points.

9. The method of claim 1, wherein the generating an arc radiotherapy plan based on a plurality of target beams in the plurality of target beam sets includes:
obtaining a plurality of pieces of dose distribution data corresponding to the plurality of target beams in the plurality of target beam sets; and
determining a dose deposition matrix based on the plurality of pieces of dose distribution data, and determining a plurality of monitor units corresponding to the plurality of target beams in the arc radiotherapy plan based on the dose deposition matrix, and a tissue weighting factor and a dose threshold corresponding to the critical structure.

10. An apparatus for generating an arc radiotherapy plan, comprising:
a storage unit configured to store a computer program; and
a processing unit configured to invoke and execute the computer program to implement a method for generating an arc radiotherapy plan, wherein the method for generating the arc radiotherapy plan comprises:
obtaining a plurality of reference beam sets corresponding to a plurality of scanning points in a target volume;
for a reference beam in one of the plurality of reference beam sets, obtaining a structure contribution of the reference beam corresponding to a critical structure and a target volume contribution of the reference beam corresponding to the target volume, and determining an importance factor of the reference beam based on the structure contribution and the target volume contribution;
obtaining a plurality of target beam sets corresponding to the plurality of scanning points based on a plurality of importance factors corresponding to a plurality of reference beams, the plurality of reference beam sets, and a complexity control parameter;
generating the arc radiotherapy plan based on a plurality of target beams in the plurality of target beam sets, wherein the arc radiotherapy plan includes beam energy and monitor units for a plurality of control points within an arc angle range; and
controlling a particle accelerator to deliver the plurality of target beams to the target volume based on the beam energy and the monitor units.

11. The apparatus of claim 10, wherein in the method implemented by the processing unit, obtaining a plurality of target beam sets corresponding to the plurality of scanning points based on a plurality of importance factors corresponding to the plurality of reference beams, the plurality of reference beam sets, and a complexity control parameter includes:
constructing a particle source selection function based on the plurality of importance factors, the plurality of reference beam sets, and the complexity control parameter, and performing an optimization solving operation on the particle source selection function to obtain the plurality of target beam sets corresponding to the plurality of scanning points.

12. The apparatus of claim 10, wherein in the method implemented by the processing unit, the determining an importance factor of the reference beam based on the structure contribution and the target volume contribution includes:
for the reference beam,
determining a first contribution based on the structure contribution of the reference beam corresponding to the critical structure, wherein the first contribution reflects a damage risk of the reference beam to the critical structure;
determining a second contribution based on the target volume contribution of the reference beam corresponding to the target volume, wherein the second contribution reflects a therapeutic benefit of the reference beam to the target volume; and
determining the importance factor of the reference beam based on a relationship where the importance factor is negatively correlated with the first contribution and the importance factor is positively correlated with the second contribution.

13. The apparatus of claim 11, wherein in the method implemented by the processing unit, the constructing a particle source selection function based on the plurality of importance factors, the plurality of reference beam sets, and the complexity control parameter includes:
for a reference beam in one of the plurality of reference beam set, obtaining beam spot data corresponding to a scanning point corresponding to the reference beam in the reference beam set; and
constructing the particle source selection function based on the plurality of importance factors, the plurality of reference beam sets, a plurality of pieces of beam spot data, and the complexity control parameter.

14. The apparatus of claim 13, wherein the particle source selection function satisfies the following conditions:
the particle source selection function reflects a combined influence of three parts, and the three parts include a first part, a second part, and a third part, wherein:
the first part uses the importance factor as a treatment utility indicator, reflecting a total treatment utility of selected reference beams, and the first part is configured to prioritize high-efficacy beams;
the second part uses the beam spot data as a precision error indicator, reflecting a total precision error of the selected reference beams, and the second part is configured to reduce a selection probability of beams with large spot sizes; and
the third part uses a count of beams as an allocation balance indicator, reflecting an allocation balance of the selected reference beams, and the third part is configured to limit beam overload in a local region; and
a constraint condition of the particle source selection function includes:
for a scanning point, a count of selected beams of the scanning point being determined based on the complexity control parameter and a count of reference beams in a reference beam set of the scanning point, wherein a selection variable is a binary variable, and the complexity control parameter is a positive integer.

15. The apparatus of claim 14, wherein the method implemented by the processing unit further comprises:
determining evaluation data corresponding to the arc radiotherapy plan based on a preset evaluation parameter;
adjusting, in response to determining that the evaluation data does not satisfy a preset evaluation condition, the complexity control parameter of the particle source selection function; and
repeating a process for constructing the particle source selection function based on the plurality of importance factors, the plurality of reference beam sets, and an adjusted complexity control parameter, and obtaining an optimized arc radiotherapy plan until the evaluation data satisfies the preset evaluation condition.

16. The apparatus of claim 11, wherein the particle source selection function further includes an energy selector option, and the energy selector option characterizes a count of selected energy layers.

17. The apparatus of claim 11, wherein in the method implemented by the processing unit, the performing an optimization solving operation on the particle source selection function to obtain the plurality of target beam sets corresponding to the plurality of scanning points includes:
obtaining a plurality of priorities corresponding to a plurality of selection items in the particle source selection function; and
performing the optimization solving operation on the particle source selection function step-by-step based on the plurality of priorities to obtain the plurality of target beam sets corresponding to the plurality of scanning points.

18. The apparatus of claim 10, wherein in the method implemented by the processing unit, the generating an arc radiotherapy plan based on a plurality of target beams in the plurality of target beam sets includes:
obtaining a plurality of pieces of dose distribution data corresponding to the plurality of target beams in the plurality of target beam sets; and
determining a dose deposition matrix based on the plurality of pieces of dose distribution data, and determining a plurality of monitor units corresponding to the plurality of target beams in the arc radiotherapy plan based on the dose deposition matrix, and a tissue weighting factor and a dose threshold corresponding to the critical structure.

19. An electronic device, comprising:
at least one processor, and
a memory communicatively connected to the at least one processor, wherein
the memory stores a computer program executable by the at least one processor, and the computer program, when executed by the at least one processor, causes the at least one processor to perform the method for generating the arc radiotherapy plan of claim 1.
